(19) United States
(12) Patent Application Publication
(10) Pub. No.: US 2025/0352346 A1
(43) Pub. Date: Nov. 20, 2025

(54) IMPLANT SYSTEM AND METHOD OF PLACING THE IMPLANT SYSTEM

(71) Applicant: TriMed, Inc., Santa Clarita, CA (US)

(72) Inventor: Robert J. Medoff, Kailua, HI (US)

(21) Appl. No.: 19/207,769

(22) Filed: May 14, 2025

Related U.S. Application Data
(60) Provisional application No. 63/647,194, filed on May 14, 2024.

(51) Int. Cl. A61F 2/30; A61B 17/72; A61F 2/38

(52) U.S. Cl. CPC A61F 2/30749; A61B 17/7233; A61F 2002/3809; A61F 2002/3827

(57) ABSTRACT

An implant system and method of placing the implant system. The implant system has: an intramedullary part with a stem configured to be directed into a bone/bone part; and a terminal part which in turn cooperates with another mechanical or anatomical part. Connectors on the intramedullary part and terminal part cooperate so that the connectors thereon can be placed in a starting position and thereafter relatively moved within a first path to be engaged. A fixation component is advanced into an opening defined by the intramedullary part and/or terminal part to produce a wedging action that generates forces between cooperating surfaces/parts on the intramedullary part and terminal part applied in directions other than along the line of the first path of the terminal part and the intramedullary part.

### BACKGROUND OF THE INVENTION

### Field of the Invention

This invention relates to medical implants cooperating between separate bones/bone parts and, more particularly, to an implant system including at least an intramedullary part directed into an intramedullary canal on one bone/bone part, that is connected to a terminal part that is directly or indirectly engaged with another bone/bone part.

### Background Art

There are occasions in orthopedic surgery where it is desired to provide an implant system that has component parts which can be assembled to create a unitary implant structure, that is utilized by itself or in conjunction with one or more additional implant components to afford different desired functionalities.

By way of example, and without limitation, many joint replacement systems are designed to provide separate parts that allow the surgeon to independently adjust the size of an articular replacement part, the length of a neck between the articular replacement part and a stem on an intramedullary part, adjust the offset of the articular part from the stem, and vary the size and shape of the stem itself. Providing a system where individual parts for specific attributes are assembled into a single structure avoids the need for a large inventory of fixed configuration implants that collectively offer anticipated permutations of each criteria.

There are different known methods that are used to couple one component to another component, each of which has certain benefits and limitations. One standard design pairs a conically-shaped trunnion piece on one component that couples to a nearly matching concave conical receptacle on a second component. If designed properly with consideration of the type of material, nominal, controlled mismatch between the angular geometry of the trunnion segment to the angular geometry of the receptacle allows the two components to lock together. Although this “Morse taper” design is widely used in connecting two components, it does require a certain minimum length for the coupling interface to be functional and can limit this design to regions where this length can be accommodated-such as hip and shoulder replacements. Additionally, a Morse taper arrangement requires that an intramedullary part be sufficiently displaced away from adjacent osseous and soft tissues in order to provide sufficient room to line up and advance the components in assembly. This requirement is not always possible or may cause soft tissue damage.

Another method of coupling components uses a cam or ratcheting design, such as disclosed in U.S. Pat. No. 9,687,350. This type of system works best where one component has a modulus of elasticity that is different from that of the other or allows sufficient structural deformation so that one part can lock into another. When both parts are made from blocks of the same material, application of this method is limited.

There are occasions where the anatomy at the local site of application makes it difficult to directly couple one part to another using the above methods. For instance, during radial head replacement it can be very difficult to displace the shaft of the radius sufficiently away from the elbow joint so that enough room is provided to allow a radial head component to be axially assembled with a prepositioned intramedullary part on a stem, such as with a Morse taper arrangement. For this type of surgical procedure, it is advantageous to have the radial head part assemble on to the intramedullary part by sliding it from the side, resulting in reduced surgical exposure and soft tissue release.

Existing designs to provide a side loading system for coupling and locking two parts together, such as for radial head replacements, have been based on some variation of a tongue-in-groove sliding connection. This allows one part to engage the other and slide along a predefined path to a final desired relationship. These designs typically fix the two parts in the final operative position with a simple bolt or screw—commonly advanced in a path that is transverse to the line of the path of relative sliding movement between the radial head part and the intramedullary part.

However, since a nominal difference in tolerance between the two components is needed to allow the two components to be assembled together, micromotion between the components occurs. Furthermore, the passage for the bolt/screw needs to be slightly larger than the diameter of the bolt to allow it to be advanced during insertion; this can result in micromotion between the structure bounding this passage and the bolt. Because of these differences in tolerance, significant stress concentration where the bolt traverses the junctions of the parts is produced; this stress concentration coupled with micromovement between the parts and/or bolt/screw can eventually result in fatigue failure of the bolt/screw and potentially catastrophic failure of the implant for the patient.

### SUMMARY OF THE INVENTION

It is an objective of the invention to provide a design that distributes loads across substantial contact areas by creating a camming effect as a fixation bolt is advanced, as to thereby generate a clamping action along a greater length of the bolt (than the interface between the components) and create frictional engagement of the two components over a substantial surface area. This may reduce stress concentration and risk of fatigue failure of the bolt.

In one form, the invention is directed to a method of placing an implant system. The method includes the steps of: a) obtaining an implant system comprising an intramedullary part, a terminal part, and a fixation component, the intramedullary part having: i) a stem with a length and configured to be directed into an intramedullary canal in a bone; and ii) a connector, the terminal part having a connector that is configured to cooperate with the connector on the intramedullary part, the connector on the terminal part and the connector on the intramedullary part configured to be engaged by aligning the connector on the terminal part and the connector on the intramedullary part in a starting relationship and thereafter moving at least one of the intramedullary part and terminal part relative to the other of the intramedullary part and terminal part along a first path extending transversely to the length of the stem on the intramedullary part to thereby place the connectors on the terminal part and intramedullary parts in a coupled relationship, wherein the terminal part and the intramedullary part are in an operative relationship and in the operative relationship are blocked from being fully separated from each other, by relative movement of the terminal part and the intramedullary part other than along the first path; b) with the connectors on the terminal part and intramedullary parts in the coupled relationship, advancing the fixation component into an opening defined by at least one of the terminal part and intramedullary part and thereby producing a wedging action that causes at least one surface on one of the terminal part and intramedullary part to be urged in a direction different from a direction along the first path, against at least one surface on the other of the terminal part and intramedullary part to thereby generate a frictional holding force between the at least one surface on the one of the terminal part and intramedullary part and the at least one surface on the other of the terminal part and intramedullary part that resists relative movement between the intramedullary part and terminal part along the first path; and c) directing the stem into an intramedullary canal in a first bone.

In one form, the first path is a substantially straight path. The step of advancing the fixation component into an opening involves advancing the fixation component in a substantially straight line that is transverse to a line of the first path.

In one form, the substantially straight line is substantially orthogonal to the line of the first path.

In one form, the opening into which the fixation component is advanced is defined cooperatively by the intramedullary part and the terminal part.

In one form, the connectors on the terminal part and the intramedullary part are configured to make a keyed connection with each other whereby the terminal part and intramedullary part are consistently guided in relative movement between the starting relationship and the coupled relationship.

In one form, the connector on one of the terminal part and the intramedullary part is in the form of a rail that moves guidingly in a complementary slot on the other of the terminal part and the intramedullary part as the terminal part and intramedullary part are relatively moved to be changed from the starting relationship into the coupled relationship.

In one form, the fixation component has a shaft with an axis and a threaded portion that is threadably engaged within a portion of the opening defined by the terminal part.

In one form, the fixation component shaft has a tapered wedging portion spaced axially from the threaded portion of the shaft of the fixation component.

In one form, the step of producing a wedging action involves bearing the wedging portion on the shaft of the fixation component against a part of the intramedullary component as the fixation component is advanced into the opening to thereby urge at least a part of the terminal part relative to the intramedullary part in a direction along the length of the stem.

In one form, the at least one surface on the one of the terminal part and intramedullary part is a first surface on the terminal part. The at least one surface on the other of the terminal part and intramedullary part is a first surface on the intramedullary part. The first surface on the terminal part faces oppositely to the first surface on the intramedullary part. The first surface on the terminal part and the first surface on the intramedullary part each face in a lengthwise direction relative to the length of the stem of the intramedullary part.

In one form, the at least one surface on the one of the terminal part and intramedullary part consists of first and second surfaces on the terminal part. The at least one surface on the other of the terminal part and intramedullary part consists of first and second surfaces on the intramedullary part. The first surfaces on the terminal part and intramedullary part and second surfaces on the terminal part and intramedullary part are urged against each other at spaced first and second locations as the fixation component is advanced into the opening.

In one form, the first and second locations are diametrically opposite with respect to the axis of the shaft of the fixation component.

In one form, the first and second surfaces on the intramedullary part are substantially flat and reside at a same radial side of the shaft of the fixation component.

In one form, the implant system has a top and bottom. The terminal part is at the top of the implant system. The wedging action produced by advancing the fixation component into the opening causes the terminal part to be moved downwardly relative to the intramedullary part.

In one form, the implant system has a top and bottom. The terminal part is at the top of the implant system. The wedging action produced by advancing the fixation component into the opening causes the terminal part to be moved upwardly relative to the intramedullary part.

In one form, the at least one surface on the at least one of the terminal part and intramedullary part is a first surface on the terminal part. The at least one surface on the other of the terminal part and intramedullary part is a first surface on the intramedullary part. The first surfaces on the terminal part and intramedullary part are substantially flat, face oppositely to each other, and reside in planes at acute angles to the length of the stem.

In one form, the first and second locations are spaced along the axis of the shaft of the fixation component.

In one form, the first surfaces on the terminal part and the intramedullary part are substantially flat and parallel to one plane and the second surfaces on the terminal part and intramedullary part are substantially flat and parallel to another plane. The one and another plane are angled relative to each other.

In one form, the step of producing a wedging action involves causing a wedging force of progressively increasing magnitude to be generated as the fixation component is advanced into the opening.

In one form, the at least one surface on the one of the terminal part and intramedullary part is a first surface on the terminal part. The at least one surface on the other of the terminal part and intramedullary part is a first surface on the intramedullary part. The first and second surfaces are each substantially flat and in facially confronting relationship with each other.

In one form, at least one of the first and second surfaces is textured.

In one form, the implant system has a lengthwise axis. The step of advancing the fixation component involves causing the fixation component to move so that the axis of the fixation component shaft shifts along the lengthwise axis of the implant system.

In one form, a portion of the opening defined by the intramedullary part has an entry end and an exit end for the advancing fixation component. The radially enlarged wedging portion of the fixation component shaft has a tapered outer surface. The step of advancing the fixation component involves causing the tapered outer surface to become wedged at the entry end of the portion of the opening defined by the intramedullary part.

In one form, the step of advancing the fixation component involves causing the tapered outer surface to engage a surface portion with a complementary shape at the entry end of the portion of the opening defined by the intramedullary part.

In one form, a portion of the opening defined by the intramedullary part has an entry end and an exit end for the advancing fixation component. The radially enlarged wedging portion of the fixation component shaft has a tapered outer surface. The step of advancing the fixation component involves causing the tapered outer surface to become wedged at a location between the entry end and the exit end of the portion of the opening defined by the intramedullary part.

In one form, the step of advancing the fixation component involves causing at least a part of the threaded portion of the shaft to be directed into and fully through a portion of the opening defined by the intramedullary part.

In one form, the first bone is a radius bone. The terminal part of the implant system defines a replacement head on the radius bone.

In one form, the step of placing the connector on the terminal part and intramedullary part in a coupled relationship is performed with the stem on the intramedullary part directed into the intramedullary canal on the radius bone and without requiring movement of the radius bone significantly away from an ulnar bone.

In one form, the frictional holding force generated between the at least one surface on the one of the terminal part and intramedullary part and the at least one surface on the other of the terminal part and intramedullary part resists translational movement between the intramedullary part and terminal part.

In one form, the frictional holding force generated between the at least one surface on the one of the terminal part and intramedullary part and the at least one surface on the other of the terminal part and intramedullary part resists turning movement between the intramedullary part and terminal part.

In one form, the tapered wedging portion on the fixation component shaft cooperates with a tapered wedging portion on at least one of the terminal part and intramedullary part to produce the wedging action.

In one form, the implant system has a top and bottom. The terminal part is at the top of the implant system. The wedging action produced by advancing the fixation component causes a part of the terminal part to turn one of upwardly and downwardly relative to the intramedullary part.

In one form, the invention is directed to an implant system having an intramedullary part, a terminal part, and a fixation component. The intramedullary part includes: a) a stem with a length and configured to be directed into an intramedullary canal in a first bone with the intramedullary part in an operative position; and b) a connector. The terminal part is configured to engage another bone/bone part with the terminal part and intramedullary part in operative relationship with each other. The terminal part has a connector configured to cooperate with the connector on the terminal part. The connector on the terminal part and the connector on the intramedullary part are configured to be engaged by aligning the connector on the terminal part and the connector on the intramedullary part in a starting relationship and thereafter moving at least one of the intramedullary part and terminal part relative to the other of the intramedullary part and terminal part along a first path extending transversely to the length of the stem on the intramedullary part to thereby place the connectors on the terminal part and intramedullary parts in a coupled relationship wherein the terminal part and the intramedullary part are in an operative relationship wherein: i) an opening is defined by at least one of the intramedullary part and the terminal part; and ii) the intramedullary part and terminal part are blocked from being fully separated from each other by relative movement of the terminal part and the intramedullary part other than along the first path. The implant system is configured so that with the terminal part and intramedullary part in the operative relationship, the fixation component can be advanced into the opening in a second path so as to produce a wedging action that causes at least one surface on one of the intramedullary part and terminal part to be forcibly urged against at least one surface on the other of the intramedullary part and terminal part in a direction different from a direction along the first path to thereby generate a frictional holding force between the at least one surface on the at least one surface on the one of the intramedullary part and the terminal part and the at least one surface on the other of the intramedullary part and the terminal part that resists relative movement between the intramedullary part and terminal part along the first path.

In one form, the first path is substantially straight.

In one form, the second path is substantially straight and transverse to a line of the first path.

In one form, the opening into which the fixation component is advanced is defined cooperatively by the intramedullary part and the terminal part.

In one form, the connectors on the terminal part and the intramedullary part are configured to make a keyed connection with each other whereby the terminal part and intramedullary part are consistently guided in relative movement between a relationship, wherein the connectors on the intramedullary part and terminal part are in the starting relationship, and the coupled relationship.

In one form, the connector on one of the terminal part and the intramedullary part is in the form of a rail that moves guidingly in a complementary slot on the other of the terminal part and the intramedullary part as the connectors on the terminal part and intramedullary part are relatively moved to be changed from the starting relationship into the coupled relationship.

In one form, the fixation component has a shaft with an axis and a threaded portion that is threadably engaged within a portion of the opening defined by the terminal part.

In one form, the fixation component shaft has a radially enlarged wedging portion spaced axially from the threaded portion of the shaft of the fixation component.

In one form, the implant system is configured so that the wedging action is produced by bearing the wedging portion on the shaft of the fixation component against a part of the intramedullary component as the fixation component is advanced into the opening to thereby urge at least a part of the terminal part relative to the intramedullary part in a direction along the length of the stem.

In one form, the at least one surface on the one of the terminal part and intramedullary part is a first surface on the terminal part and the at least one surface on the other of the terminal part and intramedullary part is a first surface on the intramedullary part. The first surface on the terminal part faces oppositely to the first surface on the intramedullary part. The first surface on the terminal part and the first surface on the intramedullary part each face in a lengthwise direction relative to the length of the stem of the intramedullary part.

In one form, the at least one surface on the one of the terminal part and intramedullary part comprises first and second surfaces on the terminal part. The at least one surface on the other of the terminal part and intramedullary part comprises first and second surfaces on the intramedullary part. The implant system is configured so that the first surfaces on the terminal part and intramedullary part and second surfaces on the terminal part and intramedullary part are urged against each other at spaced locations as the fixation component is advanced into the opening.

In one form, the first and second locations are diametrically opposite with respect to the axis of the shaft of the fixation component.

In one form, the first and second surfaces on the intramedullary part are substantially flat and reside at a same radial side of the shaft of the fixation component.

In one form, the implant system has a top and bottom. The terminal part is at the top of the implant system. The implant system is configured so that the wedging action produced by advancing the fixation component into the opening causes the terminal part to be moved downwardly relative to the intramedullary part.

In one form, the implant system has a top and bottom. The terminal part is at the top of the implant system. The implant system is configured so that the wedging action produced by advancing the fixation component into the opening causes the terminal part to be moved upwardly relative to the intramedullary part.

In one form, the at least one surface on the at least one of the terminal part and intramedullary part comprises a first surface on the terminal part. The at least one surface on the other of the terminal part and intramedullary part comprises a first surface of the intramedullary part. The first surfaces on the terminal part and intramedullary part are substantially flat, face oppositely to each other, and reside in planes at acute angles to the length of the stem.

In one form, the first and second locations are spaced along the axis of the shaft of the fixation component.

In one form, the first surfaces on the terminal part and the intramedullary part are substantially flat and parallel to one plane. The second surfaces on the terminal part and intramedullary part are substantially flat and parallel to another plane. The one and another planes are angled relative to each other.

In one form, the at least one surface on the one of the terminal part and intramedullary part comprises a first surface on the terminal part. The at least one surface on the other of the terminal part and intramedullary part comprises a first surface on the intramedullary part. The first surfaces are each substantially flat and in facially confronting relationship with each other.

In one form, at least one of the first surfaces is textured.

In one form, a portion of the opening defined by the intramedullary part has an entry end and an exit end for the advancing fixation component. The radially enlarged wedging portion of the fixation component shaft has a tapered outer surface that becomes wedged at the entry end of the portion of the opening defined by the intramedullary part as the fixation component is advanced in the opening.

In one form, a portion of the opening defined by the intramedullary part has an entry end and an exit end for the advancing fixation component. The radially enlarged wedging portion of the fixation component shaft has a tapered outer surface that becomes wedged at a location between the entry end and the exit end of the portion of the opening defined by the intramedullary part as the fixation component is advanced in the opening.

In one form, the wedging action causes the at least one surface on the one of the intramedullary part and terminal part to be forcibly urged directly against the at least one surface on the other of the intramedullary part and terminal part.

### BRIEF DESCRIPTION OF THE DRAWINGS

FIG. 1 is a schematic representation of an implant system, according to the present invention, with an intramedullary part to be placed in an intramedullary canal of a bone/bone part and a terminal part connected to the intramedullary part and in turn cooperating with a separate part/bone part;

FIG. 2 is a flow diagram representation of a method of placing an implant using the implant system in FIG. 1;

FIG. 3 is a schematic representation of bones at an elbow, a representative site at which the present invention may be practiced, including a radius bone with a radial head;

FIG. 4 is a view as in FIG. 3, wherein a fracture has occurred at the radial head;

FIG. 5 is a view as in FIGS. 3 and 4, wherein a fragment produced by the fracture in FIG. 4 is removed;

FIG. 6 is a view as in FIG. 5 and showing the radius being drawn away from an ulna bone to produce a space adequate to allow a conventional reamer to be used to prepare the radial intramedullary canal to accept a prosthetic stem;

FIG. 7 is a view as in FIG. 6 wherein a conventional prosthetic stem has been directed into the bone canal;

FIG. 8 is a view as in FIG. 7 wherein a conventional prosthetic radial head is being assembled to the stem in FIG. 7;

FIG. 9 is an exploded, perspective view of parts of one exemplary form of replacement system, according to the present invention, including an intramedullary part, in an operative position on a bone, such as a radial bone, and a terminal part defining a replacement head and joinable with the intramedullary part;

FIG. 10 is a view as in FIG. 9 with the intramedullary part and terminal part in operative relationship;

FIG. 11 is a view of the components in FIG. 10 from a different perspective;

FIG. 12 is a cross-sectional view of the terminal part and intramedullary part in operative relationship and taken along line 12-12 of FIG. 10;

FIG. 13 is a view as in FIG. 10 wherein a fixation component is aligned to be advanced into an opening defined by the terminal part and the intramedullary part;

FIG. 14 is a view as in FIG. 13 wherein the fixation component has been initially advanced into the opening defined by the terminal part and intramedullary part;

FIG. 15 is a cross-sectional view of the implant system taken along line 15-15 of FIG. 14;

FIG. 16 is a view as in FIG. 15 wherein the fixation component is further advanced into the opening defined by the terminal part and intramedullary part;

FIG. 17 is a schematic depiction of cooperating rail and slot connections between the intramedullary part and terminal part;

FIGS. 18-20 are schematic depictions of relationships between the anchoring base and fixation component in different states during assembly corresponding successively to FIGS. 13, 15, and 16;

FIG. 21 is a fragmentary view showing a modified form of a wedging portion of an intramedullary part as it cooperates with the fixation component as seen in FIGS. 18-20;

FIG. 22 is an exploded perspective view of cooperating intramedullary and terminal parts on a modified form of implant system, according to the invention;

FIG. 23 is a view as in FIG. 22 wherein the parts have been placed in an operative relationship;

FIG. 24 is a view as in FIG. 23 with a fixation component aligned to be directed into an opening defined cooperatively by the intramedullary and terminal parts;

FIG. 25 is an elevation view of the implant system as in FIG. 24 with the fixation component threadably engaged;

FIG. 26 is a view as in FIG. 25 with the implant system turned 90° about a vertical axis;

FIG. 27 is a view as in FIGS. 25 and 26 with the implant system turned an additional 90° about the vertical axis from the FIG. 26 orientation;

FIG. 28 is an enlarged, fragmentary, cross-sectional view of the implant system taken along line 28-28 of FIG. 25;

FIG. 29 is a view as in FIG. 28 wherein the fixation component has been further advanced;

FIG. 30 is a view as in FIGS. 28 and 29 wherein the fixation component has been fully seated.

FIG. 31 is a view corresponding to that in FIG. 28 and showing a modified form of implant system, according to the invention;

FIG. 32 is a view as in FIG. 31 wherein a fixation component has been advanced further from the FIG. 31 position;

FIG. 33 is a view as in FIGS. 31 and 32 wherein the fixation component has been fully inserted;

FIG. 34 is a view as in FIG. 28 and showing a further modified form of implant system, according to the invention; and

FIG. 35 is a view as in FIG. 34 wherein the fixation component has been fully inserted.

### DETAILED DESCRIPTION OF THE PREFERRED EMBODIMENT

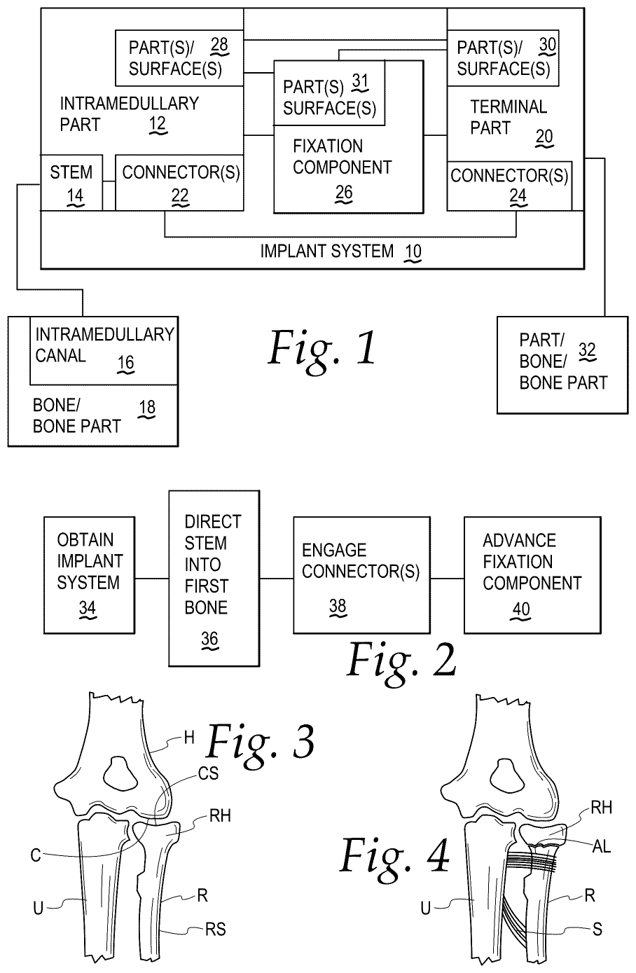

An implant system, according to the present invention, is shown schematically at 10 in FIG. 1. The implant system 10 consists of an intramedullary part 12, having a stem 14 configured to be directed into an intramedullary canal 16 on a bone/bone part 18, that engages a terminal part 20.

The terminal part 20 and the intramedullary part 12 are placed in operative relationship by aligning connectors 22, 24, respectively on the intramedullary part 12 and terminal part 20, in a starting relationship and thereafter moving at least one of the parts 12, 20 relative to the other of the parts 12, 20 along a first path extending transversely to the length of the stem 14 to thereby place the connectors 22, 24 in a coupled relationship, wherein the terminal part 20 and the intramedullary part 12 are in the operative relationship and, in the operative relationship, are blocked from being fully separated from each other by relative movement of the terminal part 20 and intramedullary part 12 other than along the aforementioned first path.

The implant system 10 further includes a fixation component 26. At least one of the terminal part 20 and intramedullary part 12 defines an opening into which the fixation component 26 is advanced with the connectors 22, 24 in the coupled relationship and the intramedullary part 12 and terminal part 20 in the operative relationship. The fixation component 26 maintains the operative relationship of the parts 12, 20 and creates a unitary implant structure with a central axis between a top and bottom thereof, with the designations “top” and “bottom” arbitrarily selected and not being limiting in terms of where the implant system 10 is used or how it is oriented.

The intramedullary part 12, the terminal part 20, and the fixation component 26 are configured so that advancing the fixation component 26 into the aforementioned opening produces a wedging action that causes at least one part/surface 28 on the intramedullary part 12 to be urged, in a direction different from a direction along the first path, against at least one part/surface 30 on the terminal part 20 to thereby generate a frictional holding force between at least one cooperating part/surface pair 28, 30 that resists relative movement between the intramedullary part 12 and terminal part 20, particularly along the first path with the intramedullary part 12 and terminal part 20 in operative relationship.

This wedging action additionally causes at least one part/surface 31 on the fixation component 26 to be forcibly urged against at least one part/surface 28 on the intramedullary part 12 and/or at least one part/surface 30 on the terminal part 20. This tends to unify the relationship between the fixation component 26 and the intramedullary part 12 and/or the terminal part 12 between which the parts/surfaces 30, 31 and/or parts/surfaces 30, 28 interact. At the same time, compression forces generated between the parts/surfaces 30, 31 and/or parts/surfaces 31, 28, including those acting in directions transverse to the first path, increase resistance to turning of the fixation component 26 with the fixation component 26 having a threaded form.

Within the schematic depiction of the implant system 10, it is contemplated that the wedging action causes the parts/surfaces 30 on the terminal part 20 to be urged either: a) directly against the parts/surfaces 28 on the intramedullary part 12; or b) indirectly against the parts/surfaces 28 on the intramedullary part 12. The indirect force transmission may be effected through one or more separate elements that transmit holding forces between the parts/surfaces 30 on the terminal part 20 and the parts/surfaces 28 on the intramedullary part 12. In one anticipated design, the holding forces acting between the intramedullary part 12 and the terminal part 20 are transmitted through the fixation component 26 that performs as such a separate element. This occurs by reason of the interaction of the parts/surfaces 28, 30, respectively on the intramedullary part 12 and fixation component 26, and the parts/surfaces 30, 31, respectively on the terminal part 20 and the fixation component 26.

The schematic depiction of components in FIG. 1 is intended to encompass virtually an unlimited number of different forms of the components depicted and their interactions.

The bone/bone part 18 to which the intramedullary part 12 is connected may be any bone/bone part, with a specific use of the implant system 10, as described hereinbelow, being exemplary in nature only.

Further, the terminal part 20 may cooperate with another mechanical part, a bone, or a bone part, as collectively schematically depicted at 32 in FIG. 1.

The connectors 22, 24 may have any configuration which allows them to move guidingly against each other between the aforementioned starting relationship and the coupled relationship. The fixation component 26 may take numerous forms, as may the portions of the intramedullary part 12 and terminal part 20 that cooperate therewith and with each other, to maintain and enhance the connection that results from changing the connectors 22, 24 from the starting relationship into the coupled relationship.

The schematic depiction of the connectors 22, 24 and their interaction is intended to encompass virtually an unlimited number of different forms and cooperating relationships, including ones wherein relative movement to effect engagement may occur in straight and non-straight paths.

For purposes of explanation only, the implant system 10 will be described based on a radial head implant arthroplasty. To those skilled in the art it can be appreciated that based on the generic system design as shown in FIG. 1 and described above, the invention can be applied to any application wherein a terminal part is connected to an intramedullary part so as to create a unitary structure that is maintained through a fixation component and that in turn cooperates with any other part-being it a man-made or an anatomical part.

Regardless of the particular application and structure, a method of placing the implant system 10 can be carried out as shown in flow diagram form in FIG. 2.

As shown at block 34, an implant system as depicted schematically in FIG. 1 is obtained.

As shown at block 36, the stem 14 on the intramedullary part 12 is directed into an intramedullary canal on a first bone.

As shown at block 38, the connectors 22, 24, respectively on the intramedullary part 12 and terminal part 20, are engaged to place the intramedullary part 12 and terminal part 20 in operative relationship.

As shown at block 40, with the intramedullary part 12 and terminal part 20 in operative relationship, the fixation component 26 is advanced into place to maintain the operative relationship of the parts 12, 20.

Before describing in detail the structure and operation of the implant system 10, one exemplary site for use of the implant system 10 will be described, with reference to FIGS. 3-8, again emphasizing that this particular site and application is exemplary in nature only.

In FIG. 3 the elbow joint location is depicted whereat the humerus H, radius R, and ulna U bones interact. The focus herein is on the radial head RH on the radial shaft RS which has a concave articular surface CS that cooperates with an articular surface on the capitellum C on the humerus H.

As seen in FIG. 4, the radius R is constrained against movement laterally away from the ulna U by, among other structures, angular ligaments AL and a syndesmosis S.

FIG. 4 depicts a condition wherein the radial head RH has been fractured, making healing impractical and the use of a prosthetic radial head appropriate.

FIG. 5 through FIG. 8 sequentially depict the procedure described in the Background Art portion hereinabove, which employs the Morse taper concept.

As shown in FIG. 5, the radial head fragment F is separated from the site.

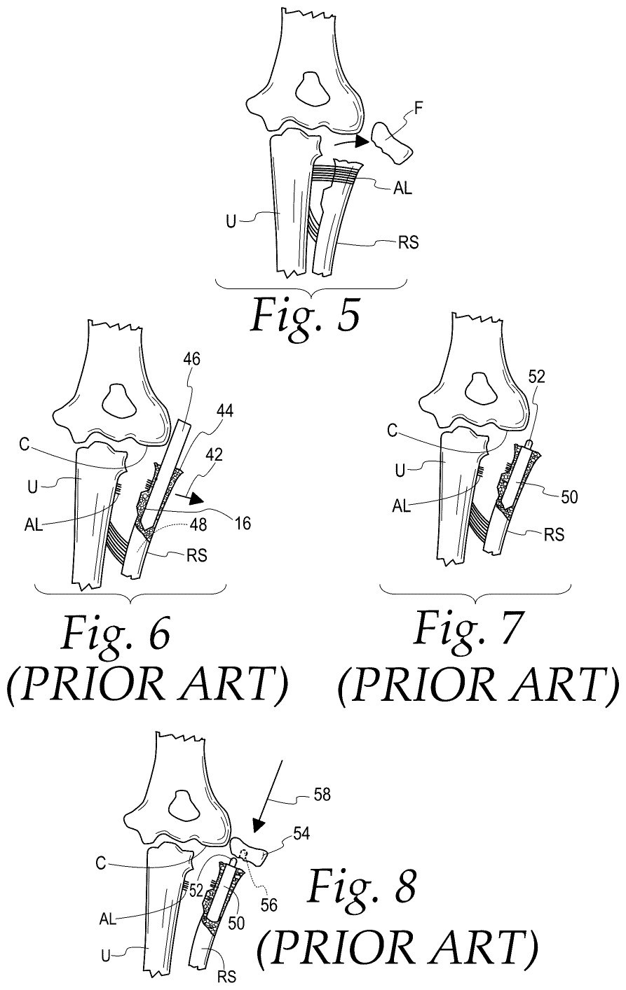

As shown in FIGS. 5-7, annular ligaments AL are severed, which allows the radial shaft portion RS, remaining after separation of the fragment F, to be moved in the direction of the arrow 42 laterally away from the ulna U and capitellum C. The separation must be such that the exposed radius surface 44 is spaced far enough away from the capitellum C that a reamer 46 can be advanced into the ulnar intramedullary canal 16 to create a receptacle for a conventional stem 50 with a trunnion 52, as shown in FIGS. 7 and 8.

The depicted prosthetic radial head replacement 54 has a concave receiving bore 56 for the trunnion 52. As seen in FIG. 8, the remaining radial shaft portion must be moved significantly away from the ulna U adequately to allow the replacement head 54 to clear the capitellum C and the trunnion 52 to be aligned under the bore 56 for placement. Once this alignment is achieved, the replacement head is driven in the direction of the arrow 58 to seat the trunnion 52 in the bore 56.

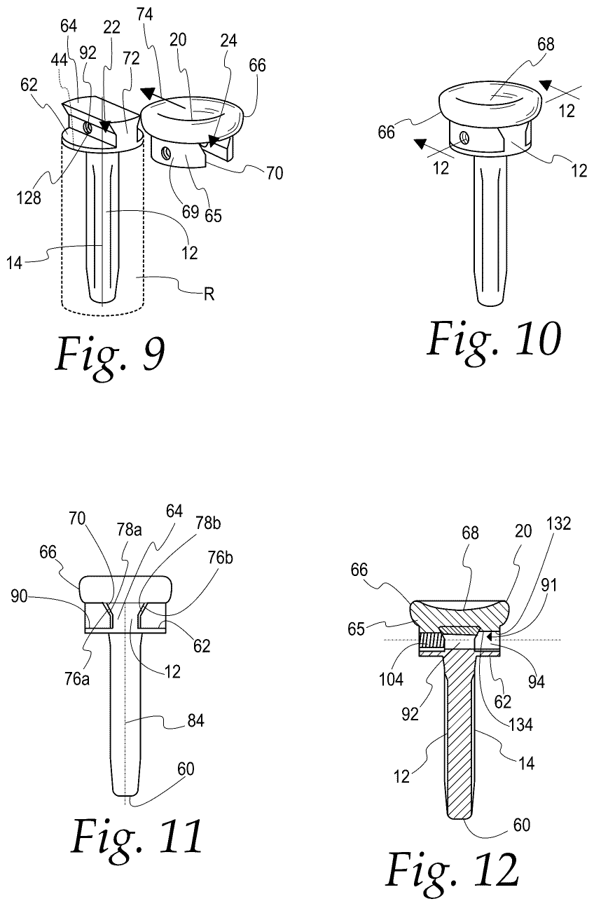

One exemplary form of the implant system 30 and a method of using the same will now be described with reference to FIGS. 9-20.

The exemplary implant system 10 consists of the intramedullary part 12 with the stem 14, the terminal part 20 that as shown defines a replacement head, and the fixation component 26.

The stem 14 is shown with a curved length with a tapered and rounded distal end 60 for assisted penetration into the intramedullary radius bone canal. The proximal region of the intramedullary part 12 has an annular flange 62 which abuts the exposed radius surface 44, defined after separation of the fragment F, with the intramedullary part 12 driven into its fully seated/operative position on the schematically depicted radius bone R, as seen in FIG. 9.

In this embodiment, the connector 22 is defined partially by a rail 64 with an expanded “Y” shape, as seen clearly in FIG. 11. The rail 64 projects up from the annular flange 62 that also makes up part of the connector 22.

The terminal part 20 has a body 65 with a stepped diameter including a larger diameter portion defining an upper annular rim 66 that surrounds a concave surface 68 that cooperates with a surface on the capitellum C in the same manner that the concave articular surface on the radial head fragment F did.

The smaller diameter portion of the body 65 defines a “neck” 69 that determines the projecting height of the articular surface. The terminal part 20 may be offered with interchangeable units, as with different neck sizes, to adapt to different site geometry.

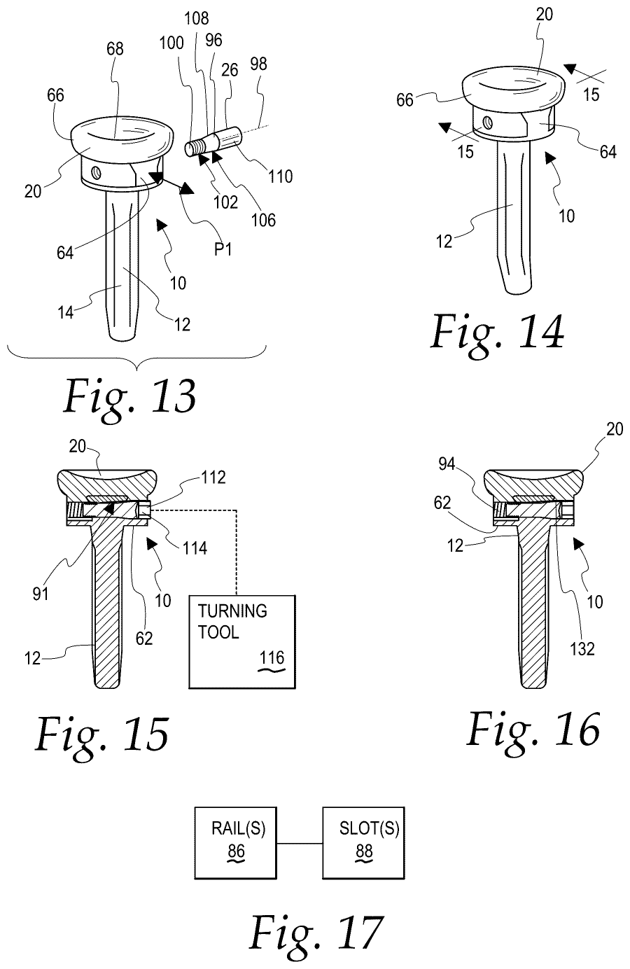

In this embodiment, the connector 24 on the terminal part 20 that cooperates with the connector 22 is a slot arrangement that is complementary in shape to the rail 64, whereby one end of a slot 70 is alignable with an end 72 of the rail 64, as shown in FIG. 9, wherein the connectors 22, 24 are in a spaced but aligned starting relationship whereupon the terminal part 20 can be advanced guidingly in the direction of the arrow 74. As this occurs, the rail end 72 enters the slot 70, whereupon continued translational advancement of the terminal part 20 causes the terminal part 20 to consistently move guidingly in a first path P1 in a first direction, as indicated by the arrow in FIG. 13, ultimately into the position in FIGS. 10-16, wherein the connectors 22, 24 assume a coupled relationship and the terminal part 20 and intramedullary part 12 are in their operative relationship.

The parts/surfaces 76,78, described below, correspond respectively to the parts/surfaces 28, 30, shown schematically in FIG. 1, with the parts/surfaces 79, described below, corresponding to the parts/surfaces 31, shown schematically in FIG. 1.

A first part/surface 76a on the rail 64 cooperates with a first part/surface 78a bounding the slot 70. A second part/surface 76b on the rail 64 cooperates with a second part/surface 78b bounding the slot 70.

In this embodiment, all of the cooperating parts/surfaces 78a, 76a; 78b, 76b are flat, with the cooperating parts/surfaces 78a, 76a; 78b, 76b being in facially confronting relationship. However, the flat surface configuration is not required.

The rail and slot arrangement keys the terminal part 20 against movement relative to the intramedullary part 12 other than in the aforementioned first path P1 in the first direction and in the first path P1 in a direction opposite to the first direction once the terminal part 20 and intramedullary part 12 have realized their operative relationship. Thus, to fully separate the terminal part 20 and intramedullary part 12, they must be relatively moved along the first path P1.

While a specific rail and slot arrangement has been depicted, it should be understood that any keying arrangement might be utilized that allows the terminal part 20 to be guidingly moved relative to the intramedullary part 12 along a path that is transverse to the lengthwise axis 84 of the stem 14—with the lengthwise axis 84 considered herein to be substantially parallel to the length of the radius bone R into which the stem 14 is advanced with the intramedullary part 12 in its operative position.

While the stem 14 is slightly curved, it will be treated herein as having a straight length, identified by the axis 84. A more curved stem 14 is also considered herein to have a straight length.

As shown schematically in FIG. 17, any combination of one or more rails 86 may be used to cooperate with one or more slots 88 to define the connectors 22, 24 and guide relative movement between the terminal part 20 and intramedullary part 12. The location of the rails 86 and slots 88—on the intramedullary part 12 and terminal part 20—is of course interchangeable. It should also be understood that while the first path is depicted as straight and substantially orthogonal to the stem axis 84, the rail(s) 86 and slot(s) 88 may guide movement of the replacement head 32 in a non-straight path that is not precisely orthogonal to the axis 84. An angular range of paths transverse to the axis 84 is contemplated. The schematic depiction of FIG. 17 is intended to encompass all such variations. What is desirable about the establishment of the path of movement for the terminal part 20 is that it limits the clearance volume required between the operatively positioned stem structure and the capitellum C, as seen clearly in FIG. 8.

In this embodiment, the cooperating parts/surfaces 78a, 76a make up a first cooperating pair, and the cooperating parts/surfaces 78b, 76b make up a second cooperating pair, with all partis/surfaces 78a, 76a, 78b, 76b residing in planes, each angled with respect to the stem axis 84—in the depicted form making an acute angle therewith. As depicted, the planes of the first parts/surfaces 78a, 76a are also angled with respect to the planes of the second parts/surfaces 78b, 76b.

With the terminal part 20 and intramedullary part 12 in the operative relationship of FIGS. 10-16, the terminal part 20 becomes captured along the axis 84 between the parts/surfaces 76a, 76b and the upwardly facing surface 90 on the annular flange 62.

With the terminal part 20 and intramedullary part 12 in the operative relationship, the aforementioned opening 91 for the fixation component 26 is defined cooperatively by the terminal part 20 and intramedullary part 12, with it being understood that the design could be such that only one of the terminal part 20 and intramedullary part 12 defines the opening 91.

With the terminal part 20 and intramedullary part 12 in the operative relationship, a part 92 of the opening 91 defined by the intramedullary part 12 registers with a part 94 of the opening 91 defined by the terminal part 20. The parts 92, 94 thus cooperatively define the opening 91 that defines a second path P2 for the fixation component 26 that is advanced into the opening 91. As depicted, the path P2 is straight and substantially orthogonal to the first path P1, with the first path P1 identified by the double-headed arrow in FIG. 13. This orthogonal relationship, however, is not a requirement.

The fixation component 26 has a shaft 96 with an axis 98. A portion of the shaft 96 adjacent a leading end 100 of the fixation component 26 has a threaded length/region at 102 configured to engage threads 104 bounding a part of the opening part 94 that is radially to one side of the stem axis 84.

The shaft 96 has a wedging portion at 106 defined by a tapered outer surface portion 108 of the shaft 96 that progressively increases in diameter away from the threaded region 102 over a length L2 of the shaft 96 and transitions to a constant diameter portion at 110 that extends over a length L3 up to a trailing shaft end 112 at which a fitting 114 is defined to be engaged by a turning tool 116. The wedging portion 106 is at an angle α with respect to the axis 98.

That portion of the opening part 94, that is diametrically opposite to where the threads 104 are located, has a diameter slightly greater than that of the constant diameter region 110 of the shaft 96 to facilitate guided advancement of the shaft region 110. In a broad sense, the design of the implant system 10 is such that once the terminal part 20 and intramedullary part 12 are placed in the operative relationship, advancement of the fixation component 26 into the opening 91 through the cooperating thread arrangement effects relative movement between at least parts/surfaces of the terminal part 20 and intramedullary part 12 such that at least one surface/part 76 on the terminal part 20 and at least one part/surface 78 on the intramedullary part 12 are caused to be forcibly urged, in directions different from a direction along the first path P1, either directly or indirectly against each other, which increases a frictional force therebetween that resists movement of the terminal part 20 relative to the intramedullary part 12, and particularly in either direction along the aforementioned first path P1. This frictional engagement resists relative movement in directions other than parallel to the first path P1. This frictional force generation through a wedging action may be accomplished in many different ways, within the generic depiction in FIG. 1. Details of the depicted exemplary form will now be explained further below.

In the depicted form, the fixation component 26 is symmetric around the shaft axis 98 along its entire length. The opening part 92 on the intramedullary part 12 has an axis 118 which aligns with the axis 98 of the shaft 96 of the fixation component 26 as the fixation component 26 is initially advanced into the opening part 92, having first traversed the portion of the opening part 94 diametrically opposite to the threads 104.

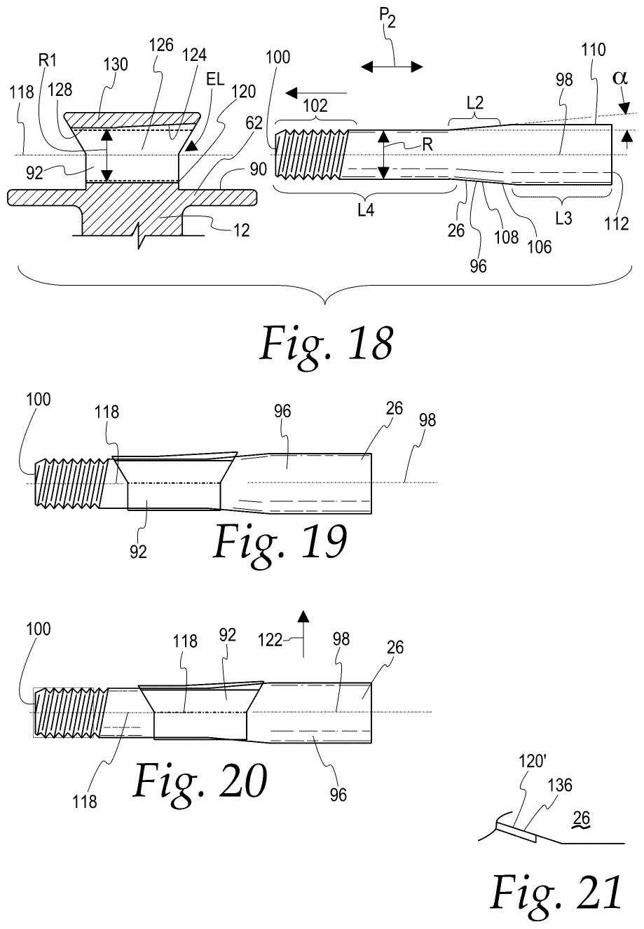

The leading region of the fixation component 26 over the shaft length L4 has a radius R that is slightly less than the radius R1 of the opening part 92, allowing it to pass through without restriction to engage the threaded length 102 of the shaft 96 with the threads 104 on the terminal part 20. The outer diameter of the opening part 92 is spaced slightly above the surface 90 of the annular flange 62 as seen clearly in FIG. 18. As the fixation component 26 is turned through the tool 116, the fixation component 26 continues to advance. The length L4 with the constant radius R moves through the opening part 92 while maintaining coincidence between the axis 98 of shaft 96 of the fixation component 26 and the axis 118 of the opening part 92. This axially aligned relationship is depicted in FIG. 19.

As this advancement continues, the tapering length L2 of the outer surface 108 of the shaft 96, that progressively increases in diameter in a trailing direction, encounters an edge 120 at an entry location EL on the opening part 92. Continued advancement of the fixation component 26 produces a wedging action between the tapered outer surface 108 and the edge 120 that tends to shift the fixation component 26 in the direction of the arrow 122 in FIG. 20, that is upwardly along the length of the radius bone R and the stem axis 84. A discrete recess/cutout 124 is formed in a wall surface 126, bounding the opening part 92, and is configured to be at least nominally complementary to the outer surface 108 along the length L2. As the fixation component 26 continues to advance, the fixation component 26 shifts in the direction of the arrow 122 in FIG. 20 while maintaining a substantially parallel alignment between the axes 118, 98. A void 128 is formed in the wall 130 on the intramedullary part 12 that bounds the opening part 92 to allow the upstream length of the fixation component 26 to shift upwardly while maintaining the parallel relationship between the axes 98, 118. As seen in FIG. 20, the axis 98 shifts to be slightly above the axis 118.

The length L3 of the surface 108 on the fixation component 26 bears against a downwardly facing region 132 of a surface 134 bounding the opening part 94, diametrically opposite to the location of the threads 104. Accordingly, as the fixation component 40 is advanced and shifts in the direction of the arrow 122, the wedged fixation component 26 in turn wedges the terminal part 20 upwardly relative to the intramedullary part 12. This causes the surfaces/parts 78a, 78b on the terminal part 20 to be urged upwardly and forcibly against the oppositely facing surfaces/parts 76a, 76b on the intramedullary part 12. This enhances a frictional connection between these parts 78a, 76a; 78b, 76b over a substantial area which resists, among other relative movement, relative translational movement between the terminal part 20 and intramedullary part 12 along the first path P1. Accordingly, with the terminal part 20 and intramedullary part 12 in operative relationship, forces tending to translate the terminal part 20 relative to the intramedullary part 12 may be positively resisted by the frictional holding forces between the parts 78a, 76a; 78b, 76b. Thus, micromotion between the terminal part 20 and intramedullary part 12 can be substantially eliminated, at the same time reducing the likelihood of failure of the fixation component 26 since it may not be acted upon by any significant forces, resulting from relative movement between the intramedullary part 12 and terminal part 20, tending to shear the fixation component 26.

With this embodiment there are surfaces/parts 76, 78 that are frictionally engaged with each other in pairs at spaced locations—in this case spaced along the axis of the fixation component 26.

The angular relationship of these cooperating surfaces/parts 76, 78 further stabilizes and rigidifies the connection between the terminal part 20 and intramedullary part 12 in multiple directions.

It should be understood that the frictional holding forces between cooperating parts/surfaces on the terminal part 20 and intramedullary part 12 may be generated by causing the camming action to urge the terminal part 20 in an opposite/downward direction relative to the intramedullary part 12, as explained in further detail below with respect to another embodiment of the invention. This is accomplished by using a substantially similar arrangement of cooperating parts/surfaces while causing the camming action to be produced so as to draw the terminal part 20 downwardly.

Further, while the axes 98, 118 are shown to be maintained in substantially parallel relationship throughout the connection of the terminal part 20 to the intramedullary part 12, the structure may be designed so that the axis 98 slightly reorients to a non-parallel relationship with the axis 118. This may result from a structure that causes the actuator component 26 to skew relative to the intramedullary part 12 rather than simply shift while maintaining constant parallel relationship between the axes 98, 118.

Further, while the wedging action is shown to cause the relative movement between the terminal part 20 and intramedullary part 12 to occur along the axis 84, the wedging may cause the relationship of the terminal part 20 and intramedullary part 12 to be changed by causing the terminal part 20 to skew, twist, pivot, etc.

The alignment of the interacting parts/surfaces 76, 78 in each pair may be changed to strategically control interaction and thereby vary frictional forces generated therebetween. While matched facial interaction is disclosed, changing to a non-matched relationship is contemplated to vary frictional holding forces and characteristics between the parts/surfaces 76, 78.

In an alternative, modified form, as shown in FIG. 21, the corner 120′, corresponding to the corner 120 in FIG. 18, may be modified by providing a tapered wedging surface 136 that is more complementary to the contour of the outer surface 108 of the fixation component 26 along the length L2. This avoids a sharp contact area and may facilitate smoother transitioning of the fixation component into its fully advanced position, as shown in FIG. 15.

For purposes of simplicity, the fixation component in all embodiments will be considered to be advanced in substantially a straight line path even though it may be shifted and/or angularly reoriented slightly relative to its starting line during insertion.

Similarly, while the first path P1 is shown to be straight, the first path may be non-straight so long as the movement of the terminal part 20 is in a direction transverse to the length of the stem 14, thereby obviating the need for a large volume around the capitellum C to engage the terminal part 20 and intramedullary part 12. This transverse relationship may be within a substantial angular range.

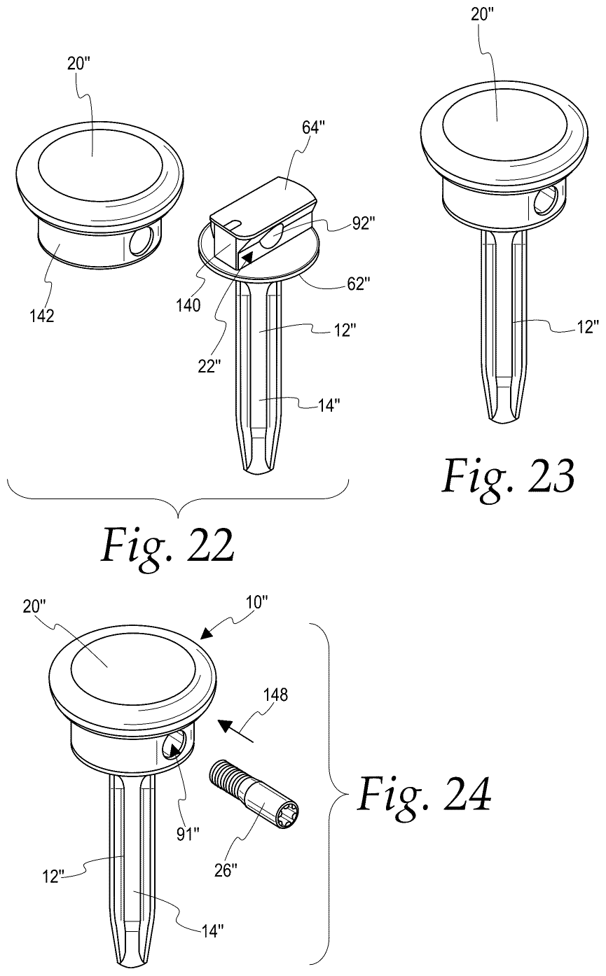

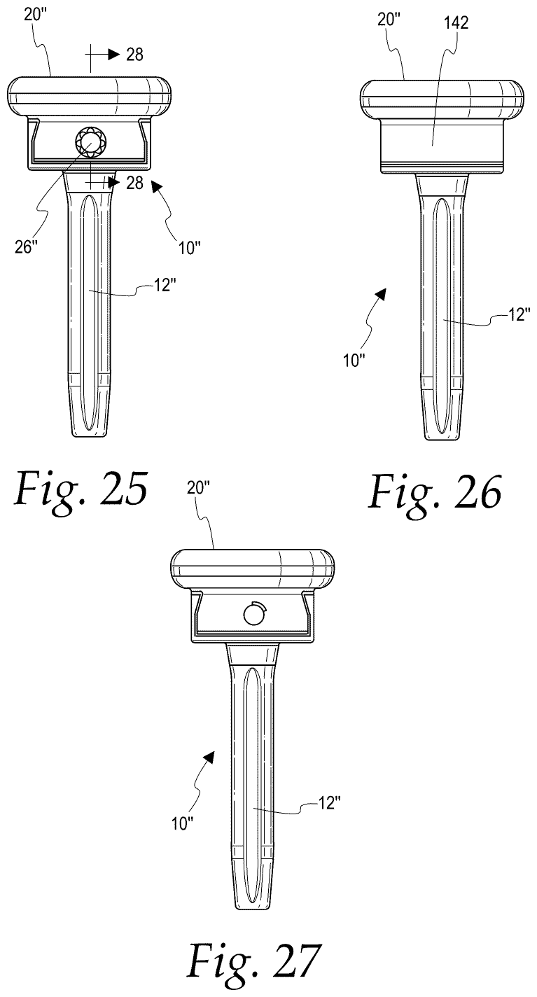

A modified, and preferred, form of implant system is shown in FIGS. 22-30 at 10″. Parts in the implant system 10″, corresponding to those in the previously described implant system 10, will be identified with corresponding reference numerals plus the addition of a “″”.

The implant system 10″ is made up of a terminal part 20″, which functions as a replacement radial head when utilized to perform the functions of a damaged or failed radial head. The implant system 10″ further includes an intramedullary part 12″ and a fixation component 26″. The intramedullary part 12″ has the same general configuration as the intramedullary part 12, including a stem 14″, a rail 64″ projecting above an annular flange 62″, and an opening portion 92″.

In this form, the rail 64″ does not extend over the full diameter of the annular flange 62″. As seen in FIG. 22, one end 140 of the rail 64″ is truncated. This accommodates a depending wall part 142 on the terminal part 20″ that abuts thereto with the intermedullary part 12″ and terminal part 20″ relatively moved into the operative relationship, to thereby consistently maintain that relationship wherein the axes of the opening parts 92″ on the intramedullary part 12″ and the opening part 94″ on the terminal part 20″, that cooperatively define the opening 91″ through which the fixation component 26″ is advanced, are aligned.

While not required, the rail 64″, and the slot 70″ on the terminal part 20″ that cooperates therewith, have substantially the same cooperating configuration as the rail 64 and slot 70 on the implant system 10.

In this embodiment, the intramedullary part 12″, terminal part 20″, and fixation component 26″ are configured so that as the fixation component 26″ is advanced so as to be fully inserted, the terminal part 20″ is cammed in a downward direction relative to the intramedullary part 12″. As a result, the enhanced frictional holding forces generated between the intramedullary part 12″ and terminal part 20″ rely on interaction of different cooperating parts/surfaces 76″, 78″.

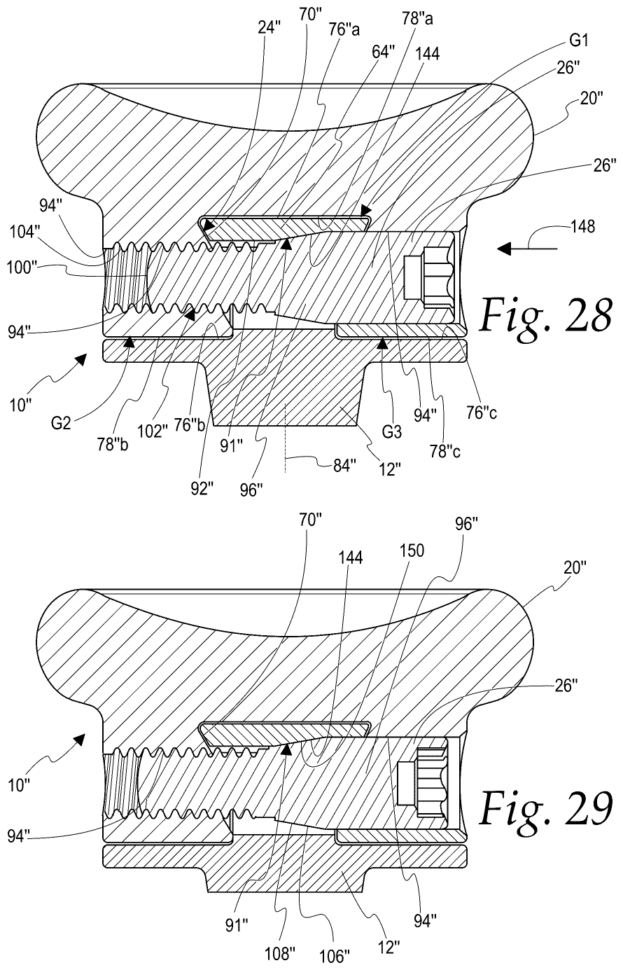

As seen in FIGS. 28-30, the upper region of the opening part 92″ has a discrete, strategically formed receptacle 144 designed to cooperate with the outer surface 108″ on the shaft 96″ of the fixation component 26″.

Connection of the intramedullary part 12″ and terminal part 20″ is carried out in the same manner as with the corresponding components in the implant system 10. That is, the connector(s) 22″ on the intramedullary part 12″ and connector(s) 24″ on the terminal part 20″ are aligned in a starting relationship and thereafter relatively moved along the aforementioned first path to place them in the coupled relationship as shown in FIGS. 23-30, wherein the intramedullary part 12″ and terminal part 20″ are in the operative relationship.

Referring to FIG. 24, the fixation component 26″ is then aligned with the opening 91″ and advanced in the direction of the arrow 148.

As shown in FIG. 28, the fixation component 26″ is advanced to the point that the threaded region 102″ at the leading end 100″ of the shaft 96″ engages the threads 104″ within the bore portion 94″ in the terminal part 20″.

To allow the guided sliding relative movement between the intramedullary part 12″ and the terminal part 20″, as they are changed from an initially spaced starting relationship into their operative relationship, a slight clearance is provided between cooperating parts/surfaces 76, 78 thereon. With the rail 64″ shown vertically centered within the cooperating slot 70″, as shown in FIG. 28, there are three different gap regions G1, G2, G3 defined with a modicum of clearance between cooperating surfaces/parts on the intramedullary part 12″ and terminal part 20″ bounding the gaps G1, G2, G3.

More specifically, the gap G1 is bounded by an upwardly facing surface/part 76″a on the intramedullary part 12″ and a downwardly facing surface part 78″a on the terminal part 20″.

The gap G2 is bounded by an upwardly facing surface/part 76″b on the intramedullary part 12″ and a downwardly facing surface/part 78″b on the terminal part 20″.

The gap G3 is bounded by an upwardly facing surface/part 76″c on the intramedullary part 12″ and a downwardly facing surface/part 78″c on the terminal part 20″.

At each gap G1, G2, G3, the paired surfaces/parts 76″, 78″ are confronting flat surfaces which face axially relative to the lengthwise stem axis 84″. This particular surface/part shape and orientation are, however, not required.

Advancement of the fixation component 26″ in the direction of the arrow 148 into the FIG. 28 position, and thereafter into the FIG. 29 position, causes: a) the outer surface 108″ of the tapered wedging portion 106″ to engage a complementarily-inclined, tapered surface portion 150 bounding the receptacle 144; and b) a progressively increasing wedging force to be generated between the outer surface 108″ and surface portion 150.

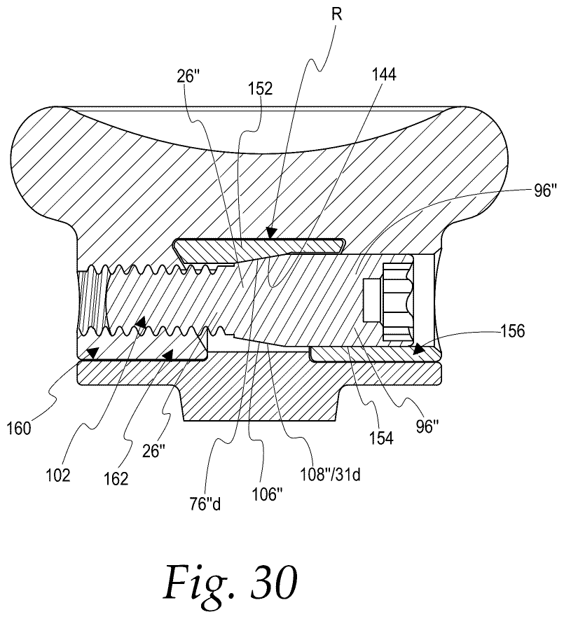

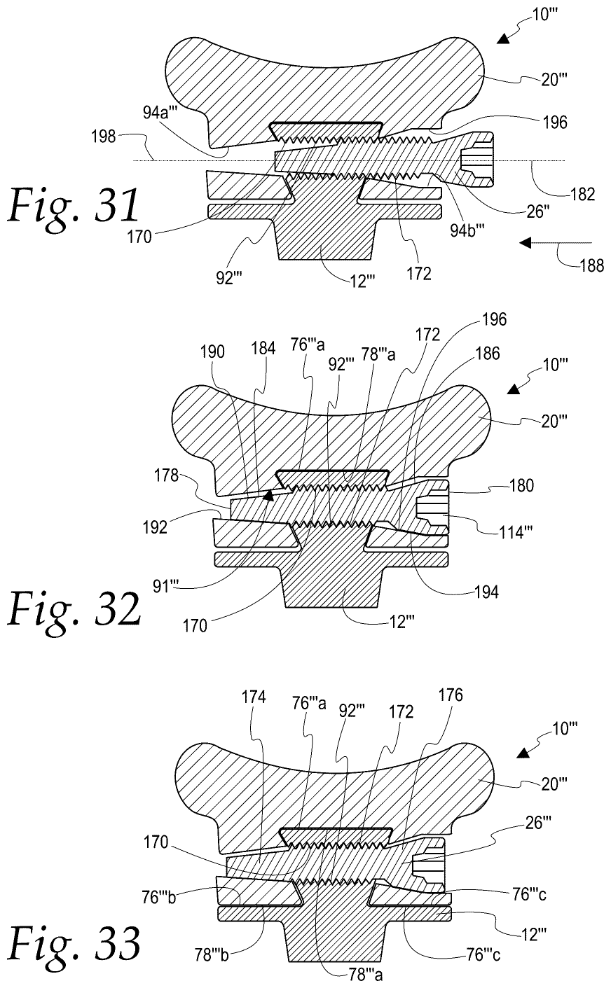

Continued advancement of the fixation component 26″ to the FIG. 30 position causes a further increased wedging force to be generated between the surface 108″ and the surface portion 150, thereby forcing the fixation component 26″ downwardly relative to the upper region 152 of the intramedullary part 12″ within the opening portion 92. As this occurs, the trailing surface region 154 of the shaft 96″ bears downwardly at one lower location 156 of the terminal part 20″, with the threaded region at 102 of the shaft 96″ bearing downwardly against the lower portion 160 of the terminal part 20″ at a second location 162 diametrically opposite to the location 156.

This interaction of components causes the fixation component 26″ to shift downwardly from its FIG. 28 position without changing its angular relationship with respect to the lengthwise axis 84″ of the intramedullary part 12″. As with the implant system 10, skewing of the fixation component 26″ is also contemplated. The terminal part 20″ in either case follows the downward shifting of the fixation component 26″.

As this occurs, the gaps G1, G2, G3 are reduced or eliminated, bringing the cooperating surface/part pairs 76″a, 78″a; 76″b, 78″b; and 76″c, 78″c forcibly into engagement with each other which creates frictional holding forces therebetween which resist relative movement between the intramedullary part 12″ and terminal part 20″, including along the first path.

As noted above, compression forces generated through the camming action at different locations between the fixation component 26″ and the terminal part 20″ and/or intramedullary part 12″ further enhance the connection between the terminal part 20″ and intramedullary part 12″.

As further noted above, the camming action produces compression forces between at least one part/surface on the fixation component 26″ that cooperates with at least one part/surface on at least one of the terminal part 20″ and intramedullary part 12″. One exemplary location is seen in FIG. 30 wherein the outer surface 108″ of the tapered wedging portion 106″ defines a part/surface 31d, as schematically depicted on the fixation component 26 in FIG. 1, that cooperates with a part/surface 76″d on the intramedullary part 12″ to generate frictional holding forces between the fixation component 26″ and the intramedullary part 12″. That is, as the fixation component 26″ is advanced, the tapered parts/surfaces 31d, 76″d interact and progressively wedge to a greater degree. Once the FIG. 30 state is realized, the region R at the top of the intramedullary part 12″ is positively captively held between portions of the fixation component 26″ and the terminal part 20″. This creates a rigid unitary connection between the intramedullary part 12″, the terminal part 20″, and the fixation component 26″ at this location, with the frictional forces, apart from resisting translational and turning movement between the intramedullary part 12″ and terminal part 20″, also resists turning of the fixation component within the opening 91.

With this embodiment, and prior embodiments, some or all engaging surfaces/parts 76, 76″; 78, 78″ may have a textured feature. A “textured” surface, as contemplated, is other than a polished surface that glides easily relative to other surfaces, and may be formed by surface roughening, providing a matte finish, etc.

With the implant system 10″, the surfaces/parts 76″, 78″ that are urged against each other are at spaced locations—in this embodiment both at diametrically opposite locations relative to the opening 91″ and at axially spaced locations therealong. The cooperating surfaces/parts 76″, 78″ at the locations of gaps G2 and G3 are at the same radial side of the shaft 96″.

In this embodiment, the wedging interaction occurs within the opening part 94″ between its entry and exit ends, whereas in the prior embodiment, the wedging occurs at the entry end.

Different configurations of surfaces/parts 76, 78, in both embodiments described above, are contemplated. Utilizing the inventive concepts, one skilled in the art could exploit the advantages described above to change the configuration and number of cooperating surfaces/parts.

In FIGS. 31-33, a modified form of implant system is shown at 10′″. Parts in the implant system 10′″ corresponding to those parts in the implant systems 10, 10″ will be identified with corresponding reference numerals with the addition of a “′″”.

In the implant system 10′″, a terminal part 20′″ and intramedullary part 12′″ are configured to be connected with each other in substantially the same manner as corresponding parts in the implant systems 10, 10″.

One of the primary differences between the implant system 10′″ and the implant systems 10, 10″ is that the fixation component 26′″ is threadably engaged with the intermedullary part 12′″.

More specifically, the opening part 92′″ has internal threads 170 that engage external threads 172 formed between an unthreaded leading length 174 on the fixation component 26″ and an unthreaded trailing length 176 thereon.

The leading length 174 tapers progressively from where the threads 172 are formed up to a leading free end 178.

The trailing length 176 tapers towards the threads 172 from a trailing free end 180. A constant diameter region is formed adjacent the free end 180 whereat a fitting 114′″ is provided for a turning tool.

In the depicted form, the fixation component 26′″ is symmetrical about its lengthwise axis 182.

The tapered leading length 174 and tapered trailing length 176 respectively define truncated conical wedging portions 184, 186, respectively.

The terminal part 20′″ has opening parts 94a′″, 94b′″ which are made with a complementary shape to the wedging portions 184, 186 to produce the appropriate camming action, as described below.

As shown in FIG. 31, the free end 178 of the fixation component 26′″ is directed through the opening part 94b′″ and the opening part 92, with advancement effected through turning with an appropriate engaged tool.

Continued advancement of the fixation component 26′″ in the direction of the arrow 188 causes the external surface 190 on the wedging part 184 to engage an internal surface 192 bounding the opening part 94a′″ at the same time that an external surface 194 on the wedging part 186 engages an internal surface 196 bounding the opening part 94b′″.

The axis 182 of the fixation component 26′″ is initially below the axis 198 of the opening 91′″. As a result, as the fixation component 26′″ is advanced in the direction of the arrow 188 from the FIG. 31 position, the tapered external surface 190 slides against the tapered internal surface 192, thereby wedging the terminal part 20′″ downwardly through a force applied on the left side thereof in FIG. 32.

At the same time, a similar camming action occurs between the internal surface 196 on the terminal part 20′″ and the external surface 194 on the fixation component 26′″ at the right side of the terminal part 20′″ in FIG. 32.

The complementary wedging action finally seats the fixation component as shown in FIG. 33, wherein the part/surface 76′″a on the intramedullary part 12′″ is forcibly drawn against the oppositely facing part/surface 78′″a. At the same time, the conically-shaped wedging portion 184 is pressed into the opening part 94a′″. A similar engagement occurs with the wedging portion 186 in the opening part 94b′″.

The parts can be relatively dimensioned so that a Morse taper type of connection is established at both openings parts 94a′″, 94b′″.

Additional parts/surfaces 76, 78 may be engaged as the fixation component 26′″ is urged downwardly to the FIG. 33 position.

More specifically, parts/surface 76′″b, 78′″b; and 76′″c, 78′″c may cooperate at opposite end regions of the fixation component 26′″.

With this embodiment, as in all embodiments, it should be understood that the cooperating parts/surfaces 76, 78, 31, all shown to be in a potentially cooperating relationship, do not necessarily have to be engaged with the implant system 10, 10″, 10′″ in its final configuration. Any one or more of such paired parts/surfaces 76, 78, 31 may engage to produce an implant system with components adequately held together using the inventive concepts.

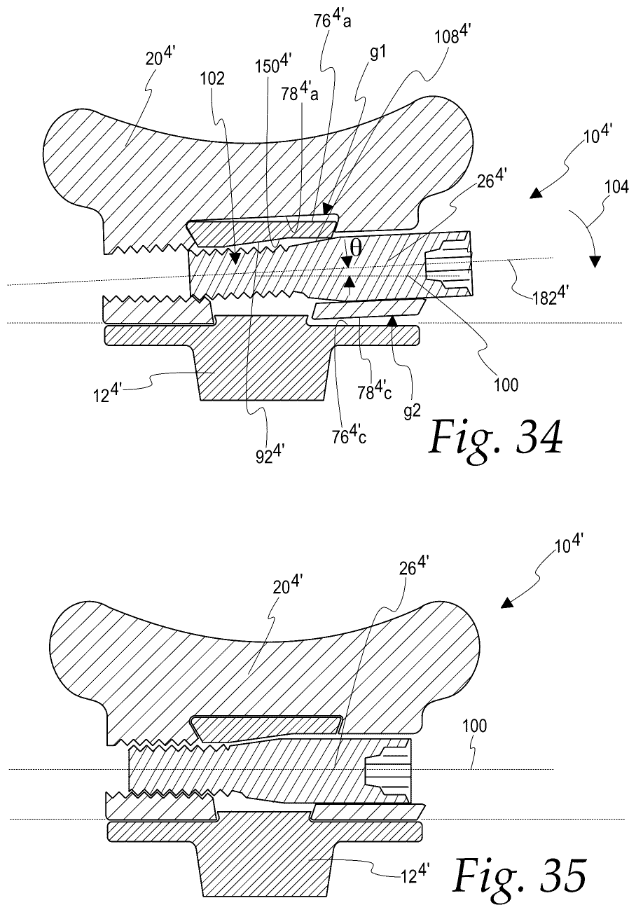

A still further form of implant system, according to the invention, is shown at 10^{4′} in FIGS. 34 and 35.

The implant system 10^{4′} uses a terminal part 20^{4′} that cooperates with an intramedullary part 12^{4′} in substantially the same manner as the corresponding parts cooperate in the previously described implant systems 10, 10″, 10′″.

In this embodiment, the relationship between the intramedullary part 12^{4′} and terminal part 20^{4′} is slightly skewed from the previously described relationship. As seen in FIG. 34, the axis 182^{4′} of the partially advanced fixation component 26^{4′} makes an angle θ with the axis 100 for the opening part 92^{4′}. This produces gap regions g1, g2—respectively between parts/surfaces 78^{4′}a, 76^{4′}a, and 76^{4′}c, 78^{4′}c.

With a threaded leading length 102 of the fixation component 26^{4′} advanced from the FIG. 34 position into the FIG. 35 position, a tapered surface 108^{4′} on the fixation component 26^{4′} bears against a tapered surface portion 150^{4′} on the intramedullary part 12^{4′}. This turns the right side of the terminal part 20^{4′} in the direction of the arrow 104, thereby closing one or both of the gaps g1, g2, whereupon the final implant system configuration is arrived at in FIG. 35.

The change between the FIG. 34 and FIG. 35 states may be effected by causing the terminal part 20^{4′} to turn, pivot, or deform in this transition.

The skewed relationship of components potentially creates a number of binding connections between parts that enhance the unitary connection of the terminal part 20^{4′}, the intramedullary part 12^{4′}, and the fixation component 26^{4′}.

Using the inventive concepts, a unitary implant system 10, 10″ can be provided with positively engaged parts that act as a unit and are not prone to relative micromovement. Mating parts/surfaces can have different interacting shapes and may be relatively dimensioned to control frictional forces generated therebetween.

The foregoing disclosure of specific embodiments is intended to be illustrative of the broad concepts comprehended by the invention.

1. A method of placing an implant system, the method comprising the steps of:
obtaining an implant system comprising an intramedullary part, a terminal part, and a fixation component,
the intramedullary part having: a) a stem with a length and configured to be directed into an intramedullary canal in a bone; and b) a connector,
the terminal part having a connector that is configured to cooperate with the connector on the intramedullary part,
the connector on the terminal part and the connector on the intramedullary part configured to be engaged by aligning the connector on the terminal part and the connector on the intramedullary part in a starting relationship and thereafter moving at least one of the intramedullary part and terminal part relative to the other of the intramedullary part and terminal part along a first path extending transversely to the length of the stem on the intramedullary part to thereby place the connectors on the terminal part and intramedullary parts in a coupled relationship, wherein the terminal part and the intramedullary part are in an operative relationship and in the operative relationship are blocked from being fully separated from each other, by relative movement of the terminal part and the intramedullary part other than along the first path;
with the connectors on the terminal part and intramedullary parts in the coupled relationship, advancing the fixation component into an opening defined by at least one of the terminal part and intramedullary part and thereby producing a wedging action that causes at least one surface on one of the terminal part and intramedullary part to be urged in a direction different from a direction along the first path, against at least one surface on the other of the terminal part and intramedullary part to thereby generate a frictional holding force between the at least one surface on the one of the terminal part and intramedullary part and the at least one surface on the other of the terminal part and intramedullary part that resists relative movement between the intramedullary part and terminal part along the first path; and
directing the stem into an intramedullary canal in a first bone.

2. The method of placing an implant system according to claim 1 wherein the first path is a substantially straight path and the step of advancing the fixation component into an opening comprises advancing the fixation component in a substantially straight line that is transverse to a line of the first path.

3. The method of placing an implant system according to claim 2 wherein the substantially straight line is substantially orthogonal to the line of the first path.

4. The method of placing an implant system according to claim 1 wherein the opening into which the fixation component is advanced is defined cooperatively by the intramedullary part and the terminal part.

5. The method of placing an implant system according to claim 1 wherein the connectors on the terminal part and the intramedullary part are configured to make a keyed connection with each other whereby the terminal part and intramedullary part are consistently guided in relative movement between the starting relationship and the coupled relationship.

6. The method of placing an implant system according to claim 5 wherein the connector on one of the terminal part and the intramedullary part is in the form of a rail that moves guidingly in a complementary slot on the other of the terminal part and the intramedullary part as the terminal part and intramedullary part are relatively moved to be changed from the starting relationship into the coupled relationship.

7. The method of placing an implant system according to claim 6 wherein the fixation component has a shaft with an axis and a threaded portion that is threadably engaged within a portion of the opening defined by the terminal part.

8. The method of placing an implant system according to claim 7 wherein the fixation component shaft has a tapered wedging portion spaced axially from the threaded portion of the shaft of the fixation component.

9. The method of placing an implant system according to claim 8 wherein the step of producing a wedging action comprises bearing the wedging portion on the shaft of the fixation component against a part of the intramedullary component as the fixation component is advanced into the opening to thereby urge at least a part of the terminal part relative to the intramedullary part in a direction along the length of the stem.

10. The method of placing an implant system according to claim 9 wherein the at least one surface on the one of the terminal part and intramedullary part comprises a first surface on the terminal part, the at least one surface on the other of the terminal part and intramedullary part comprises a first surface on the intramedullary part, the first surface on the terminal part facing oppositely to the first surface on the intramedullary part, the first surface on the terminal part and the first surface on the intramedullary part each facing in a lengthwise direction relative to the length of the stem of the intramedullary part.

11. The method of placing an implant system according to claim 9 wherein the at least one surface on the one of the terminal part and intramedullary part comprises first and second surfaces on the terminal part and the at least one surface on the other of the terminal part and intramedullary part comprises first and second surfaces on the intramedullary part, the first surfaces on the terminal part and intramedullary part and second surfaces on the terminal part and intramedullary part urged against each other at spaced first and second locations as the fixation component is advanced into the opening.

12. The method of placing an implant system according to claim 11 wherein the first and second locations are diametrically opposite with respect to the axis of the shaft of the fixation component.

13. The method of placing an implant system according to claim 11 wherein the first and second surfaces on the intramedullary part are substantially flat and reside at a same radial side of the shaft of the fixation component.

14. The method of placing an implant system according to claim 1 wherein the implant system has a top and bottom, the terminal part is at the top of the implant system, and the wedging action produced by advancing the fixation component into the opening causes the terminal part to be moved downwardly relative to the intramedullary part.

15. The method of placing an implant system according to claim 1 wherein the implant system has a top and bottom, the terminal part is at the top of the implant system, and the wedging action produced by advancing the fixation component into the opening causes the terminal part to be moved upwardly relative to the intramedullary part.

16. The method of placing an implant system according to claim 9 wherein the at least one surface on the at least one of the terminal part and intramedullary part comprises a first surface on the terminal part, the at least one surface on the other of the terminal part and intramedullary part comprises a first surface on the intramedullary part, the first surfaces on the terminal part and intramedullary part are substantially flat, face oppositely to each other, and reside in planes at acute angles to the length of the stem.

17. The method of placing an implant system according to claim 11 wherein the first and second locations are spaced along the axis of the shaft of the fixation component.

18. The method of placing an implant system according to claim 17 wherein the first surfaces on the terminal part and the intramedullary part are substantially flat and parallel to one plane and the second surfaces on the terminal part and intramedullary part are substantially flat and parallel to another plane, the one and another plane angled relative to each other.

19. The method of placing an implant system according to claim 1 wherein the step of producing a wedging action comprises causing a wedging force of progressively increasing magnitude to be generated as the fixation component is advanced into the opening.

20. The method of placing an implant system according to claim 1 wherein the at least one surface on the one of the terminal part and intramedullary part comprises a first surface on the terminal part, the at least one surface on the other of the terminal part and intramedullary part comprises a first surface on the intramedullary part, the first and second surfaces each is substantially flat, the first and second surfaces in facially confronting relationship with each other.

21. The method of placing an implant system according to claim 20 wherein at least one of the first and second surfaces is textured.

22. The method of placing an implant system according to claim 7 wherein the implant system has a lengthwise axis and the step of advancing the fixation component comprises causing the fixation component to move so that the axis of the fixation component shaft shifts along the lengthwise axis of the implant system.

23. The method of placing an implant system according to claim 8 wherein a portion of the opening defined by the intramedullary part has an entry end and an exit end for the advancing fixation component, the radially enlarged wedging portion of the fixation component shaft comprises a tapered outer surface and the step of advancing the fixation component comprises causing the tapered outer surface to become wedged at the entry end of the portion of the opening defined by the intramedullary part.

24. The method of placing an implant system according to claim 23 wherein the step of advancing the fixation component comprises causing the tapered outer surface to engage a surface portion with a complementary shape at the entry end of the portion of the opening defined by the intramedullary part.

25. The method of placing an implant system according to claim 8 wherein a portion of the opening defined by the intramedullary part has an entry end and an exit end for the advancing fixation component, the radially enlarged wedging portion of the fixation component shaft comprises a tapered outer surface and the step of advancing the fixation component comprises causing the tapered outer surface to become wedged at a location between the entry end and the exit end of the portion of the opening defined by the intramedullary part.

26. The method of placing an implant system according to claim 8 wherein the step of advancing the fixation component comprises causing at least a part of the threaded portion of the shaft to be directed into and fully through a portion of the opening defined by the intramedullary part.

27. The method of placing an implant system according to claim 1 wherein the first bone is a radius bone and the terminal part of the implant system defines a replacement head on the radius bone.

28. The method of placing an implant system according to claim 27 wherein the step of placing the connector on the terminal part and intramedullary part in a coupled relationship is performed with the stem on the intramedullary part directed into the intramedullary canal on the radius bone and without requiring movement of the radius bone significantly away from an ulnar bone.

29. The method of placing an implant system according to claim 1 wherein the frictional holding force generated between the at least one surface on the one of the terminal part and intramedullary part and the at least one surface on the other of the terminal part and intramedullary part resists translational movement between the intramedullary part and terminal part.

30. The method of placing an implant system according to claim 1 wherein the frictional holding force generated between the at least one surface on the one of the terminal part and intramedullary part and the at least one surface on the other of the terminal part and intramedullary part resists turning movement between the intramedullary part and terminal part.

31. The method of placing an implant system according to claim 8 wherein the tapered wedging portion on the fixation component shaft cooperates with a tapered wedging portion on at least one of the terminal part and intramedullary part to produce the wedging action.

32. The method of placing an implant system according to wherein the implant system has a top and bottom, the terminal part is at the top of the implant system, and the wedging action produced by advancing the fixation component causes a part of the terminal part to turn one of upwardly and downwardly relative to the intramedullary part.

33. An implant system comprising:
an intramedullary part having: a) a stem with a length and configured to be directed into an intramedullary canal in a first bone with the intramedullary part in an operative position; and b) a connector;
a terminal part configured to engage another bone/bone part with the terminal part and intramedullary part in operative relationship with each other,
the terminal part having a connector configured to cooperate with the connector on the terminal part,
the connector on the terminal part and the connector on the intramedullary part configured to be engaged by aligning the connector on the terminal part and the connector on the intramedullary part in a starting relationship and thereafter moving at least one of the intramedullary part and terminal part relative to the other of the intramedullary part and terminal part along a first path extending transversely to the length of the stem on the intramedullary part to thereby place the connectors on the terminal part and intramedullary parts in a coupled relationship, wherein the terminal part and the intramedullary part are in an operative relationship wherein: a) an opening is defined by at least one of the intramedullary part and the terminal part; and b) the intramedullary part and terminal part are blocked from being fully separated from each other by relative movement of the terminal part and the intramedullary part other than along the first path; and
a fixation component,
the implant system configured so that with the terminal part and intramedullary part in the operative relationship, the fixation component can be advanced into the opening in a second path so as to produce a wedging action that causes at least one surface on one of the intramedullary part and terminal part to be forcibly urged against at least one surface on the other of the intramedullary part and terminal part in a direction different from a direction along the first path to thereby generate a frictional holding force between the at least one surface on the at least one surface on the one of the intramedullary part and the terminal part and the at least one surface on the other of the intramedullary part and the terminal part that resists relative movement between the intramedullary part and terminal part along the first path.

34. The implant system according to claim 33 wherein the first path is substantially straight.

35. The implant system according to claim 34 wherein the second path is substantially straight and transverse to a line of the first path.

36. The implant system according to claim 33 wherein the opening into which the fixation component is advanced is defined cooperatively by the intramedullary part and the terminal part.

37. The implant system according to claim 33 wherein the connectors on the terminal part and the intramedullary part are configured to make a keyed connection with each other whereby the terminal part and intramedullary part are consistently guided in relative movement between a relationship, wherein the connectors on the intramedullary part and terminal part are in the starting relationship, and the coupled relationship.

38. The implant system according to claim 37 wherein the connector on one of the terminal part and the intramedullary part is in the form of a rail that moves guidingly in a complementary slot on the other of the terminal part and the intramedullary part as the connectors on the terminal part and intramedullary part are relatively moved to be changed from the starting relationship into the coupled relationship.

39. The implant system according to claim 38 wherein the fixation component has a shaft with an axis and a threaded portion that is threadably engaged within a portion of the opening defined by the terminal part.

40. The implant system according to claim 39 wherein the fixation component shaft has a radially enlarged wedging portion spaced axially from the threaded portion of the shaft of the fixation component.

41. The implant system according to claim 40 wherein the implant system is configured so that the wedging action is produced by bearing the wedging portion on the shaft of the fixation component against a part of the intramedullary component as the fixation component is advanced into the opening to thereby urge at least a part of the terminal part relative to the intramedullary part in a direction along the length of the stem.

42. The implant system according to claim 41 wherein the at least one surface on the one of the terminal part and intramedullary part comprises a first surface on the terminal part, the at least one surface on the other of the terminal part and intramedullary part comprises a first surface on the intramedullary part, the first surface on the terminal part facing oppositely to the first surface on the intramedullary part, the first surface on the terminal part and the first surface on the intramedullary part each facing in a lengthwise direction relative to the length of the stem of the intramedullary part.

43. The implant system according to claim 41 wherein the at least one surface on the one of the terminal part and intramedullary part comprises first and second surfaces on the terminal part and the at least one surface on the other of the terminal part and intramedullary part comprises first and second surfaces on the intramedullary part, the implant system configured so that the first surfaces on the terminal part and intramedullary part and second surfaces on the terminal part and intramedullary part urged against each other at spaced locations as the fixation component is advanced into the opening.

44. The implant system according to claim 43 wherein the first and second locations are diametrically opposite with respect to the axis of the shaft of the fixation component.

45. The implant system according to claim 43 wherein the first and second surfaces on the intramedullary part are substantially flat and reside at a same radial side of the shaft of the fixation component.

46. The implant system according to claim 33 wherein the implant system has a top and bottom, the terminal part is at the top of the implant system, and the implant system is configured so that the wedging action produced by advancing the fixation component into the opening causes the terminal part to be moved downwardly relative to the intramedullary part.

47. The implant system according to claim 33 wherein the implant system has a top and bottom, the terminal part is at the top of the implant system, and the implant system is configured so that the wedging action produced by advancing the fixation component into the opening causes the terminal part to be moved upwardly relative to the intramedullary part.

48. The implant system according to claim 41 wherein the at least one surface on the at least one of the terminal part and intramedullary part comprises a first surface on the terminal part, the at least one surface on the other of the terminal part and intramedullary part comprises a first surface of the intramedullary part, the first surfaces on the terminal part and intramedullary part are substantially flat, face oppositely to each other, and reside in planes at acute angles to the length of the stem.

49. The implant system according to claim 43 wherein the first and second locations are spaced along the axis of the shaft of the fixation component.

50. The implant system according to claim 49 wherein the first surfaces on the terminal part and the intramedullary part are substantially flat and parallel to one plane and the second surfaces on the terminal part and intramedullary part are substantially flat and parallel to another plane, the one and another planes angled relative to each other.

51. The implant system according to claim 33 wherein the at least one surface on the one of the terminal part and intramedullary part comprises a first surface on the terminal part, the at least one surface on the other of the terminal part and intramedullary part comprises a first surface on the intramedullary part, the first surfaces are each substantially flat, and are in facially confronting relationship with each other.

52. The implant system according to claim 51 wherein at least one of the first surfaces is textured.

53. The implant system according to claim 40 wherein a portion of the opening defined by the intramedullary part has an entry end and an exit end for the advancing fixation component and the radially enlarged wedging portion of the fixation component shaft comprises a tapered outer surface that becomes wedged at the entry end of the portion of the opening defined by the intramedullary part as the fixation component is advanced in the opening.

54. The implant system according to claim 40 wherein a portion of the opening defined by the intramedullary part has an entry end and an exit end for the advancing fixation component, the radially enlarged wedging portion of the fixation component shaft comprises a tapered outer surface that becomes wedged at a location between the entry end and the exit end of the portion of the opening defined by the intramedullary part as the fixation component is advanced in the opening.

55. The implant system according to claim 33 wherein the wedging action causes the at least one surface on the one of the intramedullary part and terminal part to be forcibly urged directly against the at least one surface on the other of the intramedullary part and terminal part.
